(19) United States
(12) Patent Application Publication
(10) Pub. No.: US 2025/0044263 A1
(43) Pub. Date: Feb. 6, 2025

(54) Development of methods for quantitative determination of the total and reactive polysulfides: Reactive polysulfide profiling in vegetables

(71) Applicants: University Public Corporation Osaka, Osaka (JP); Bio-Xcelerator, Inc., Tokyo (JP)

(72) Inventors: Hideshi IHARA, Osaka (JP); Shingo KASAMATSU, Osaka (JP); Satoshi TAKAGI, Tokyo (JP)

(21) Appl. No.: 18/792,548

(22) Filed: Aug. 2, 2024

Related U.S. Application Data
(60) Provisional application No. 63/517,164, filed on Aug. 2, 2023.

(51) Int. Cl. G01N 30/72; G01N 33/02

(52) U.S. Cl. CPC G01N 30/7266; G01N 33/025

(57) ABSTRACT

A method for quantitative determination of total polysulfide content (TPsC) and reactive polysulfide content (RPsC) includes a step of using liquid chromatography-electrospray ionization-tandem mass spectrometry.

### 0. BACKGROUND (INTRODUCTION)

Alliaceous vegetables, such as Allium cepa (Onions) and Allium sativum (Garlic), in addition to cruciferous vegetables, such as Brassica oleracea var. italica (Broccoli), are known for being rich in bioactive organosulfur compounds, including S-alk(en)yl-L-cysteine-sulfoxides, thiosulfates, glucosinolates, and isothiocyanates (Hill, Shafaci, Balmer, Lewis, Hodgson, Millar, & Blekkenhorst, 2022; Putnik et al., 2019). An extensive search of the literature has shown that organosulfur compounds exhibit a broad spectrum of biological activities, including antioxidant, anti-inflammatory, antimicrobial, anticancer, and car-dioprotective effects, as reviewed elsewhere (Hill et al., 2022; Miękus et al., 2020; Putnik et al., 2019). The polysulfides, including dimethyl trisulfide (DMS3), dipropyl trisulfide, diallyl trisulfide (DAS3), and diallyl tetrasulfide (DAS4) are also important organosulfur compounds that exhibit a range of biological activities (Corzo-Martínez, Corzo, & Villamiel, 2007; Tocmo, Liang, Lin, & Huang, 2015). However, the exact mechanisms of action and structure-activity relationships for these compounds remain unclear due to the fact that they are easily converted into various substances through degradation, enzymatic reactions, and chemical rearrangements during processing, storage, and digestion. Because of this instability, the quantitative and qualitative analyses of organosulfur compounds can be challenging, so there is a lack of consistent clinical evidence to support the above claims.

In terms of animal studies, recent publications have highlighted the biological importance of polysulfides. For example, the endogenous production of cysteinyl- and glutathionyl-polysulfides in mammals (e.g., humans and mice) has been confirmed using liquid chromatography mass spectrometry combined with the trapping derivatization of hydropolysulfides using electrophilic alkylating agents (Ida, Sawa, Ihara, Tsuchiya, Watanabe, Kumagai, & Akaike, 2014). Indeed, there is growing evidence to suggest that these endogenous polysulfides exhibit unique chemical properties, wherein they can act as both electrophiles and nucleophiles, thereby acting as potent antioxidants and regulators of redox signaling in mice (Akaike et al., 2017; Ida et al., 2014). Moreover, recent studies have demonstrated that these bioactive sub-stances play important roles in the regulation of mitochondrial biogenesis and sulfur respiration (Akaike et al., 2017; Marutani et al., 2021). Polysulfides have also been reported to exhibit protective effects against various diseases in humans and animals, thereby indicating their potential role in human health and disease prevention.

For the detection of organosulfur compounds in biological samples, such as vegetables, recent developments in mass spectrometry are of particular importance (Miękus et al., 2020; Putnik et al., 2019; Tocmo et al., 2015). However, the data must be interpreted with caution because alternations in the ionization and detection between individual samples may occur due to differences in the inherent chemical or physical properties of each host matrix (Zhou, Yang, & Wang, 2017). Thus, to precisely detect the polysulfide content of biological samples, we developed a quantitative polysulfide metabolomics approach based on the use of liquid chromatography-electrospray ionization-tandem mass spectrometry (LC-ESI-MS/MS) combined with a stable isotope-labeled standard dilution method (Akaike et al., 2017; Ida et al., 2014; Kasamatsu et al., 2021). These detection methods allowed us to quantify a variety of endogenous polysulfides both accurately and specifically; however, only the targeted polysulfides were detectable, and untargeted/unknown polysulfides remained undetected. This can be problematic since previous studies have shown that the polysulfide concentration and composition in Allium and its extracts are altered during processing, cooking, and storage, which can in turn influence the antioxidant activity due to its correlation with the organosulfur content (S. Kim et al., 2018; Locatelli, Nazareno, Fusari, & Camargo, 2017).

Due to the fact that organosulfur compounds, including the poly-sulfides, play a crucial role in the maintenance of human health and disease prevention, it is important to evaluate the reactive polysulfide profiles of foods by quantitatively measuring the total amounts of sulfur-containing molecules and polysulfides. In the present study, we aim to develop three novel methods to quantify total sulfur content (TSC), total polysulfide content (TPsC), and reactive polysulfide content (RPsC) (FIG. 1A). Subsequently, we aim to use these methods to analyze the reactive polysulfide profiles of various Allium and Brassica vegetables, including onions, garlic, and broccoli.

Abbreviations: CAPS, N-cyclohexyl-3-aminopropanesulfonic acid; FA, formic acid; DAS, diallyl sulfide, DAS2, diallyl disulfide; DAS3, diallyl trisulfide; DAS4, diallyl tetrasulfide; DMS, dimethyl sulfide; DMS2, dimethyl disulfide: DMS3, dimethyl trisulfide; DTT, dithiothreitol; GSH, glutathione, GS2G, oxidized glutathione disulfide; GS3G, oxidized glutathione trisulfide; GS4G, oxidized glutathione tetrasulfide; ICP-OES, inductively coupled plasma optical emission spectroscopy; LC-ESI-MS/MS, liquid chromatography-electrospray ionization-tandem mass spectrometry; RPsC, reactive polysulfide content; TPsC, total polysulfide content; Tris, tris (hydroxymethyl)aminomethane; TSC, total sulfur content; SE, standard error.

Corresponding author at: Department of Biological Chemistry, Graduate School of Science, Osaka Metropolitan University, Sakai 599-8531, Japan.

E-mail address: iharah@omu.ac.jp (H. Ihara).

### 1A. SUMMARY

A method for quantitative determination of total polysulfide content (TPsC) and reactive polysulfide content (RPsC) disclosed in this application includes a step of using liquid chromatography-electrospray ionization-tandem mass spectrometry.

### 1B. BRIEF DESCRIPTION OF THE DRAWING

FIG. 1. Schematic representation outlining the three different methodologies for reactive polysulfide profiling. (A) The total sulfur content (TSC), total polysulfide content (TPsC), and reactive polysulfide content (RPsC) were determined using an acid-circulating decomposition system followed by inductively coupled plasma optical emission spectroscopy (ICP-OES), the alkaline/reductive sulfur elimination protocol, and the N-ethylmaleimide (NEM) sulfur abstraction method, respec-tively. (B) Molecular structures of the representative organosulfur compounds analyzed in this study. The sulfane sulfur atom(s) present in the polysulfides is high lighted in yellow. DAS, diallyl sulfide, DAS2, diallyl disulfide; DAS3, diallyl trisulfide; DAS4, diallyl tetrasulfide; DMS, dimethyl sulfide; DMS2, dimethyl di-sulfide; DMS3, dimethyl trisulfide; DTT, dithiothreitol; GSH, glutathione; GS2G, oxidized glutathione disulfide; GS3G, oxidized glutathione trisulfide; GS4G, oxidized glutathione tetrasulfide; LC-ESI-MS/MS, liquid chromatography-electrospray ionization-tandem mass spectrometry.

FIG. 2. Quantification of TSC by acid-circulating decomposition system. (A) Detection of sulfur atom(s) as the sulfate ion derived from the volatile polysulfides and non-volatile glutathionyl-polysulfides. (B) The recovery (%) was calculated from the detected sulfur atom(s) shown in (A). Data are presented as the mean±standard error (SE) (n=3-6).

FIG. 3. Quantification of sulfane sulfur atom(s) present in polysulfides by alkaline/reductive sulfur elimination protocol. (A) Detection of NEM-adducts derived from GS3G by the alkaline/reductive sulfur elimination protocol. (B) Comparison of the detected liberated sulfur atom(s) from various organosulfur compound standards. Data are presented as the mean±SE (n=3-6). GS-NEM, NEM-adduct of GS moiety; NEM-S-NEM, bis-sulfur-NEM-adduct.

FIG. 4. Detection of reactive polysulfide content by NEM sulfur abstraction method. (A) Kinetic analysis of the formation of NEM-S-NEM derived from oxidized glutathionyl-polysulfides. Oxidized glutathionyl-polysulfides (GS3G and GS4G) and incubated at 37° C. in the presence of NEM for 2 h, followed by the quantification of the formed NEM-S-NEM by LC-ESI-MS/MS. GS2G (0.35 or 0.7 mM) was also analyzed as a negative control. (B) Comparative analysis of the reactivity of sulfane sulfur atom(s) in lipophilic or water-soluble polysulfides using the NEM sulfur abstraction method. Various organosulfur standards including polysulfides (1 mM) were analyzed by the same procedure as mentioned in A. Data are presented as the mean±SE (n=3-6).

FIG. 5. Correlations between the TPsC and TSC (A), and the TPsC and RPsC (B) in the vegetable specimens. Linear regression was used to compare the TSC, TPsC, and RPsC values. The values of the correlation coefficient (R2) between for (A) and (B) were determined to be 0.2142 and 0.7993, respectively.

### 2. DETAILED DESCRIPTION OF PREFERRED EMBODIMENT (MATERIALS AND METHODS)

### 2.1 Materials

The organosulfur compound standards used in this study were >95% pure, with the exception of DAS4. Dimethyl sulfide (DMS, 99%), dimethyl disulfide (DMS2, 98%), DMS3 (98%), and diallyl sulfide (DAS, 98%) were purchased from Tokyo Chemical Co., ltd. (Tokyo, Japan). Diallyl disulfide (DAS2, >98%), DAS3 (95%), and DAS4 (90%) were obtained from Sigma-Aldrich (St. Louis, MO), Cayman Chemicals (Ann Arbor, MI), and Abcam (Cambridge, UK), respectively. Glutathione (GSH, 97%) and glutathione disulfide (GS2G, 95%) were purchased from Fujifilm Wako Pure Chemical (Osaka, Japan). Oxidized glutathione trisulfide and tetrasulfide (GS3G and GS4G) were prepared as previously described (Akaike et al., 2017). N-Ethylmaleimide (NEM, >99%) and dithiothreitol (DTT, >99%) were purchased from Nacalai Tesque (Kyoto, Japan). An authentic stable isotope-labeled standard of the bis-sulfur-NEM-adduct (NEM-³⁴S-NEM-d₁₀) was prepared using stable isotope-labeled NEM (NEM-d₅) and sodium sulfide (Na³⁴S), as previ-ously reported (Kasamatsu, Kakihana, Koga, Yoshioka, & Ihara, 2020). All other chemicals and reagents were purchased from general chemical suppliers and were of the highest commercially available grade.

### 2.2 Vegetables

Twenty-two types of vegetables, including onions, garlic, and broc-coli, were analyzed in this study. All vegetables were domestically cultivated, obtained at least biologically different three specimens per a species from local supermarkets, sampled each in triplicate, and stored at −80° C. until required for analysis. To determine the water content of the individual vegetables, fresh vegetable samples (˜1 g) were freeze-dried, the dry weight was measured using an electronic balance, and the water content (%) was calculated using the following formula:

$$Water content ( \% ) = [ 1 - dry weight ( g ) \div fresh weight ( g ) ] \times 100.$$

### 2.3 Quantification of the Total Sulfur Content by Acid-Circulating Decomposition and Inductively Coupled Plasma Optical Emission Spectroscopy

For quantification of the TSC, all sulfur-containing molecules were converted into sulfate ions using an acid-circulating decomposition system (ECOPRE system; ACTAC, Tokyo, Japan), and the concentration of the formed sulfate ions was measured by inductively coupled plasma optical emission spectroscopy (ICP-OES). A schematic representation of the acid-circulating decomposition system used in this study is shown in Supplementary Fig. S1A. During this process, the lipophilic organosulfur compounds (i.e., DMS, DMS2, DMS3, DAS, DAS2, DAS3, and DAS4) were diluted in acetone to avoid any intense explosive reactions. Sub-sequently, the diluted lipophilic organosulfur standards (100 μL), GSH (˜100 mg), GS2G (˜50 mg), the water-soluble polysulfides (i.e., GS3G and GS4G, 30-100 μL), and vegetable samples (˜100 mg) were mixed with a 15.8 M aqueous solution of HNO₃ (10 mL) in a digestion vessel (ACTAC), and the digestion vessel was combined with a condensation vessel (ACTAC) containing 5% (v/v) HNO₃ (5 mL). Digestion was per-formed using multiple heating/cooling cycles on a graphite hot plate (ASONE, Osaka, Japan), as outlined in detail in Supplementary Fig. S1B. After cycling and cooling to 25° C., the samples were collected, diluted to 30 mL using a 1 M HNO₃ solution, and then diluted further to concen-trations of 1-10 ppm using the same 1 M HNO₃ solution. Solvents (i.e., acetone and water) were also prepared in the absence of standards or vegetables to give the blank samples, and the signals detected from the blank samples were subtracted from the signals obtained from the in-dividual samples. The sulfate ion concentrations in the samples were measured using ICP-OES (ICPE-9000, Shimadzu, Kyoto, Japan) and were quantified using a standard curve obtained from a sulfate ion standard (Kanto Chemical, Tokyo, Japan). The ICP-OES measurement parameters were as follows: argon gas pressure, 450±10 kPa; radio-frequency power, 1.2 kW; plasma gas flow rate, 14 L/min; carrier gas flow rate, 0.7 L/min; exposure time, 30 s; observation wavelengths, 180.731, 182.037, and 182.625 nm. The recovery (%) was calculated using the following formula:

$$Recovery ( \% ) = detected sulfate ion concentration \div theoretical sulfur concentration [ compound concentration \times number of sulfur atom ( s ) in the compound ] \times 100.$$

### 2.4 Quantification of the Total Polysulfide Content Using Alkaline/Reductive Sulfur Elimination Protocol

To quantitatively determine the TPsC, the polysulfides were decomposed by reduction under alkaline conditions, and the liberated sulfur atom(s) were captured by the alkylating agent NEM to form a stable bis-sulfur-NEM-adduct (NEM-S-NEM), which was then quantita-tively detected by LC-ESI-MS/MS analysis combined with the stable isotope-labeled standard dilution method. More specifically, the organosulfur compound standards (1 mM each, 10 μL) were mixed with a 100 mM N-cyclohexyl-3-aminopropanesulfonic acid (CAPS)-NaOH buffer (pH 11.0) containing 10 mM DTT and 25% (v/v) ethanol (190 μL total), and the mixtures were incubated at 37° C. for 1 h in a heating block incubator (NICHIHIRO, Saitama, Japan). The reaction mixtures (10 μL) were then added to a 200 mM tris(hydroxymethyl)amino-methane (Tris)-HCl buffer (pH 7.4) containing 20 mM NEM and 80% (v/v) methanol (90 μL) and incubated at 37° C. for 1 h in a heatblock incubator. After centrifugation at 20,380 g and 4° C. for 15 min, the resulting supernatant (10 μL) was diluted 10 times with a 0.1% (v/v) formic acid (FA) solution containing 0.1 μM of the authentic isotope-labeled standard, NEM-³⁴S-NEM-d₁₀. The obtained mixture was then subjected into LC-ESI-MS/MS analysis as described in Section 2.6. Buffer blanks were also prepared in the absence of standards or vegetables, and the signals detected from the blank samples were subtracted from the signals obtained from the individual samples.

For quantification of the TPsC in the vegetable samples, each spec-imen (˜10-100 mg) was homogenized with a 20-fold volume of 100 mM CAPS-NaOH buffer (pH 11.0) containing 10 mM DTT and 25% (v/v) ethanol using an Ultra-Turrax-homogenizer (Model: T10 basic, IKA, Osaka, Japan), followed by sonication (15 s, repeated three times with a 30 s interval), and the homogenates were incubated at 37° C. for 1 h. The subsequent steps for sample preparation were as those described earlier.

### 2.5 Quantification of the Reactive Polysulfide Content Using NEM Sulfur Abstraction Method

To determine the RPsC, the highly reactive sulfur atom(s) present in the polysulfides were extracted under incubation with NEM at a neutral pH, and the obtained NEM-S-NEM was detected using quantitative LC-ESI-MS/MS. More specifically, the organosulfur compound standards (0.35-1 mM each, 10 μL) were mixed with a 100 mM Tris-HCl buffer (pH 7.4) containing 10 mM NEM and 80% (v/v) methanol (190 μL), and the obtained mixtures were incubated at 37° C. for 2 h. Subsequently, the reaction was terminated by adding 650 μL of a 1% (v/v) FA solution to 150 μL of the reaction mixture. Following subsequent centrifugation at 20,380 g and 4° C. for 15 min, the resulting supernatant was diluted 10 times with a 0.1% (v/v) FA solution containing 0.1 μM of the stable isotope-labeled standard and subjected to LC-ESI-MS/MS analysis (see Section 2.6). Buffer blanks were also prepared in the absence of stan-dards or vegetables, and the signals detected from the blank samples were subtracted from the signals obtained from the individual samples.

To determine the RPsC of the vegetable samples, the various speci-mens (˜10-100 mg) were mixed with a 20-fold volume of 100 mM Tris-HCl buffer (pH 7.4) containing 10 mM NEM and 80% (v/v) methanol, homogenized using an Ultra-Turrax-homogenizer, subjected to soni-cation (15 s, repeated three times with a 30 s interval), and the ho-mogenates were incubated at 37° C. for 2 h. The subsequent steps for sample preparation were those as described earlier.

### 2.6 LC-ESI-MS/Ms

For quantification of the TPsC and RPsC, the obtained NEM-adducts were detected by LC-ESI-MS/MS analysis using the stable isotope-labeled standard dilution method, with slight modifications (Kasa-matsu et al., 2020). LC-ESI-MS/MS analysis was performed using a triple quadrupole mass spectrometer (Xevo TQD; Waters, Milford, MA, USA) coupled with an Alliance e2695 system (Waters). The NEM-adducts were separated using an Alliance e2695 system equipped with a C18 reverse-phase column (Mightysil RP-18 GP 2.0×50 mm; Kanto Chemical) with a linear gradient of solvent A (water containing 0.1% FA) and solvent B (100% methanol) (gradient=1% B at 1 min; 99% B at 4 min) at a flow rate of 0.6 mL/min. The mass spectrometer was operated in the positive mode under the following conditions: capillary voltage, 1000 V; desolvation gas (N₂) flow rate and temperature, 1000 L/h, and 500° C. The endogenous NEM-adducts and the spiked stable isotope-labeled NEM-adduct standards were simultaneously identified by multiple reaction monitoring (MRM); the MRM parameters are pro-vided in Supplementary Table S1.

### 2.7 Statistical Analysis

Data are presented as the mean±standard error (SE) of at least three independent experiments, unless otherwise specified. Linear regression was used to compare the TSC, TPsC, and RPsC values. All analyses were performed using the GraphPad Prism software (GraphPad Software, La Jolla, CA, USA). Statistical significance was set at p<0.05.

### 3. RESULTS AND DISCUSSION

### 3.1 Quantitative Determination of the TSC Based on Acid-Circulating Decomposition and ICP-OES

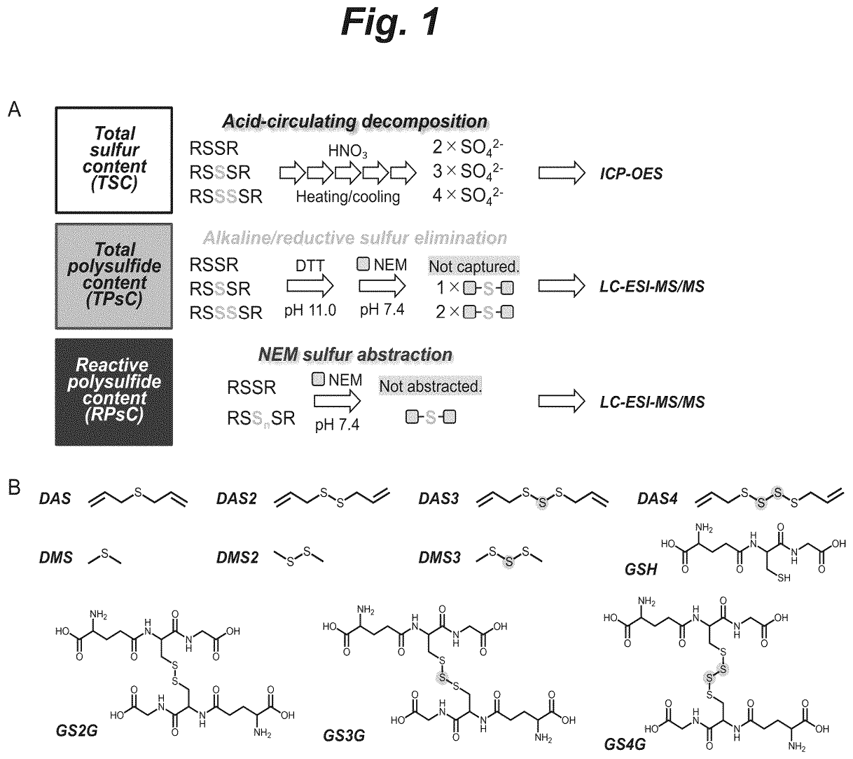

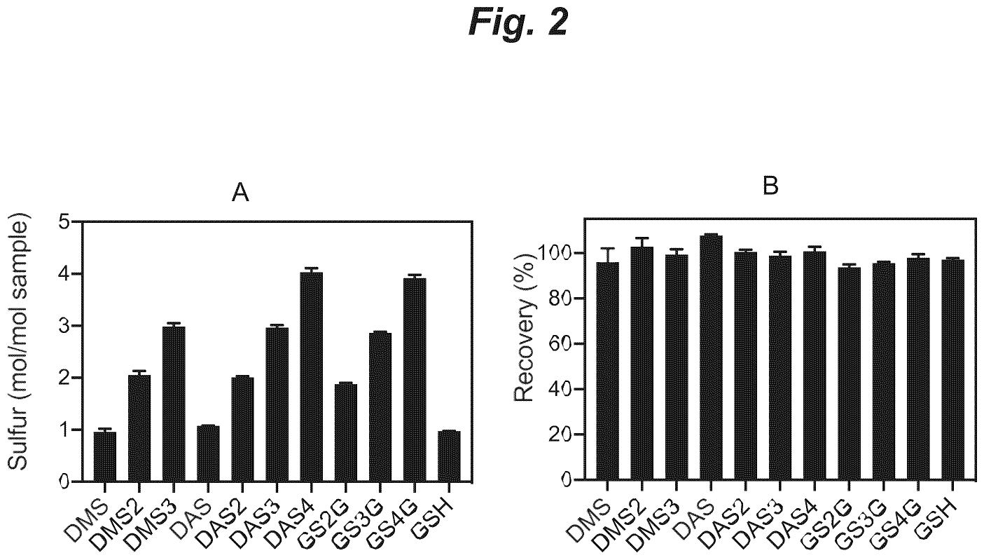

For quantification of the TSC of the vegetable samples, the sulfur-containing molecules were converted to sulfate ions by means of acid-circulating decomposition, and the concentration of the formed sulfate ions was measured by ICP-OES (FIG. 1A). Although dry ashing and wet digestion systems have been widely employed to analyze the sulfur compositions of plants (Ali, Zoltai, & Radford, 1988), the organosulfur compounds, such as the polysulfides, present in the essential oils of Allium are known to be volatile. In addition, it has been reported that sulfur atom(s) can be liberated from polysulfides under strongly acidic conditions, resulting in their release into the atmosphere as gaseous hydrogen sulfide (Høj & Møller, 1987; Ishigami et al., 2009). Taken together, these results suggest that the previously reported TSC of veg-tables may be underestimated. To address these issues, we utilized a semi-closed wet digestion system, namely an acid-circulating decom-position system. As shown in Supplementary Fig. S1A, the samples were digested by heating in HNO₃, the vapors were trapped in a condensation vessel containing 5% HNO₃, and the acid collected in the condensation vessel was returned to the digestion vessel upon cooling. Sample digestion was completed by carrying out multiple heating/cooling cy-cles. To evaluate the usefulness of this technology for measuring volatile organosulfur compounds, including polysulfides, we initially analyzed the lipophilic sulfur-containing compounds (DMS, DMS2, DMS3, DAS, DAS2, DAS3, and DAS4) and the nonvolatile sulfur-containing com-pounds (GSH, GS2G, GS3G and GS4G) as model compounds. As shown in FIG. 2A, the amount of detected sulfur increased with the number of sulfur atom(s) present in the individual chemicals. The calculated re-covery values therefore demonstrate that the detected sulfur concen-trations were identical to the theoretical values (FIG. 2B). In addition, it was confirmed that inorganic monosulfur molecules (i.e., elemental sulfur and Na₂S) were completely oxidized to sulfates by the acid-circulating decomposition system (Supplementary Fig. S2). These re-sults indicate that this semi-closed wet digestion system allows the precise measurement of not only inorganic monosulfur compounds but also organosulfur compounds, including volatile and acid-sensitive polysulfides.

### 3.2 Establishment of a Method for Quantification of the TPsC

It has been previously reported that the sulfur-sulfur bonds present in disulfides and polysulfides are sensitive to reductants (Akaike et al., 2017; Shinohara & Padis, 1936), and recent studies have revealed that the polysulfides are more susceptible to alkaline hydrolysis than the disulfides (Bogd\acute{ }andi et al., 2019; Hamid et al., 2019; Sawa et al., 2022). More specifically, the hydrolysis of polysulfides is enhanced in the presence of harsh alkylating agents, such as monobromobimane and NEM, to produce bis-sulfur-electrophile-adducts (Bogda\acute{ }ndi et al., 2019; Hamid et al., 2019; Sawa et al., 2022). Thus, the sulfane sulfur atom(s) present in polysulfides is liberated by alkaline incubation in the presence of reductants such as DTT, and the resulting species are captured by alkylating agents to form bis-sulfur-electrophile-adducts (FIG. 1A).

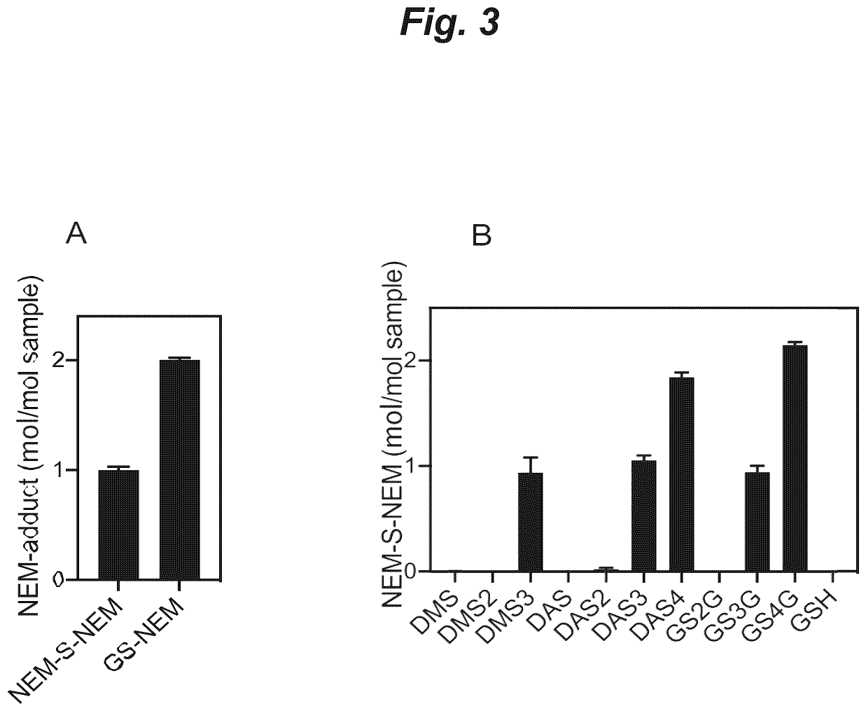

Based on these unique chemical properties of the polysulfides, we herein developed an easy, convenient, and reliable method to capture the sulfane sulfur species liberated from polysulfides, wherein NEM was used as a trapping reagent, and analysis was by quantitative LC-ESI-MS/MS, which we designated as alkaline/reductive sulfur elimination pro-tocol. Initially, we performed a model experiment using GS3G, which contains one sulfane sulfur and two GS moieties per molecule. As shown in FIG. 3A, quantitative LC-ESI-MS/MS analysis detected the NEM-S-NEM at a concentration of 1.0±0.03 mol/mol sample, while the NEM-adduct of GS moiety (GS-NEM) was detected at a concentration of 2.0±0.02 mol/mol sample. These results indicate that the polysulfide structure of GS3G was completely decomposed, and the liberated sul-fane sulfur was captured by the NEM to form NEM-S-NEM. To best of our knowledge, polysulfides that have identified so far can be mainly clas-sified into two groups: hydrophilic one (e.g., glutathione polysulfides, and protein polysulfides) and lipophilic one (e.g., dimethyl polysulfides and diallyl polysulfides). Glutathione hydropersulfide (GS2H), a hydrophilic substance, is reportedly one of the most abundant polysulfides in vivo and various polysulfides can be generated via sulfur-translation reactions from GS2H to acceptor thiols. However, it is quite difficult to isolate GS2H and use as an authentic standard because the substance is easily oxidized under aerobic conditions. Recent studies report that GS2H can be generated from GS3G and GS4G under physiological con-ditions (Ida et al., 2014; Barayeu et al., 2022). Thus, we believe that GS3G and GS4G can represent GS2H and other endogenous hydrophilic polysulfides. On the other hand, it is well known that lipophilic poly-sulfides are derived from sulfur-containing amino acids through cooking procedures with heating. Although little is known about endogenous production of lipophilic polysulfides, DAS3, DAS4, and DMS3 are reportedly one of the predominant polysulfides in essential oils obtained from Allium and Brassica vegetables (Tocmo et al., 2015). Therefore, we utilized these three compounds as a representative lipophilic poly-sulfide. In fact, the results indicate that DMS3, DAS3, DAS4, GS3G, and GS4G produced 0.9±0.1, 1.0±0.04, 1.8±0.05, 0.9±0.06, and 2.0±0.0 sulfide mol/mol sample, respectively (FIG. 3B); in other words, sul-fane sulfur(s) in both the lipophilic and hydrophilic polysulfide stan-dards were completely converted into NEM-S-NEM. We next assessed this method using various organosulfur compounds as negative control, including thiols (RSH), sulfides (RSR), and disulfides (RSSR), which contain the same side chain but no sulfane sulfur atom. It has been suggested that sulfides can be released from thiols under strongly alkaline conditions via the following reaction: RSH+2OH⁻→ROH+S⁻+H O (Olson, DeLeon, & Liu, 2014). However, as shown in FIG. 3B, our results indicated that no sulfides were detected for the RSH (GSH), RSR (DMS and DAS), and RSSR (DMS2 and GS2G) systems, thereby implying that the release of sulfide from RSH, RSR, and RSSR docs not occur under the alkaline conditions employed in the current method. These results suggest that the content of polysulfides (but not other organosulfur compounds) can be specifically detected using the alkaline/reductive sulfur elimination protocol developed herein. It should also be noted that in the case of DAS2 sample, the compound detected at a concentration of 0.002 sulfide mol/mol sample may orig-inate from diallyl-polysulfide contaminations in the standard.

### 3.3 Assessment of the Methodology for the Detection of Reactive Polysulfides

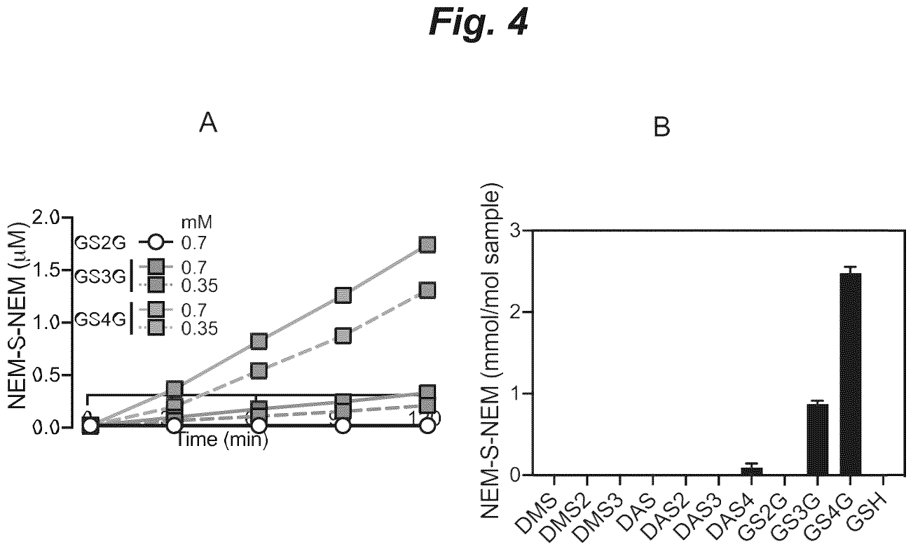

Emerging evidence from the MS-based analyses of glutathionyl-polysulfides and alkylated polysulfides indicates that strong electro-philic thiol-reactive reagents, such as monobromobimane and NEM, can react with the midchain sulfane sulfur atom(s) present in polysulfides, resulting in the formation of sulfur-electrophile-adducts, including bis-sulfur-electrophile-adducts (Akaike et al., 2017; Bogd\acute{ }andi et al., 2019; Hamid et al., 2019; Sawa et al., 2022). In addition, the reactivity of these sulfane sulfur species at neutral pH has been shown to increase with the polysulfide chain length (Hamid et al., 2019; Kasamatsu et al., 2021). In contrast to the polysulfides, RSSR is relatively stable at physiological or neutral pH values, and the bis-sulfur-electrophile-adduct is not produced by the reaction with electrophilic alkylating agents (Anderson & Berg, 1969). This indicates the unique reactivity of the polysulfides and allowed us to develop an analytical technique for determining the RPsC. More specifically, we employed NEM as an alkylating agent to extract highly reactive sulfur atom(s) from the polysulfides, and the resulting NEM-S-NEM was quantitatively detected by LC-ESI-MS/MS analysis, which we designated as NEM sulfur abstraction method. It was found that the formation of NEM-S-NEM followed pseudo-first order reaction kinetics, and so the reaction rate reflected the reactivity of the original compound (FIG. 4A). We then assessed this method using polysulfide standards, as well as other organosulfur compounds. As shown in FIG. 4B, the formation of NEM-S-NEM was observed for GS4G, GS3G, and DAS4, and the detected sulfide concentration was found to correlate with the polysulfide chain length (FIG. 4B). It should be noted here that, as shown in FIG. 3B, although the three trisulfide standards (GS3G, DAS3, and DMS3) each contained one labile sulfane sulfur atom, the reactivities of their sulfane sulfur atom were different at a neutral pH (FIG. 4B). Thus, the result of the NEM sulfur abstraction method demonstrates that the molecular structure, including the side chain and the number of sulfane sulfur atom, of the polysulfide affects the sulfane sulfur reactivity. In addition, it has reported that the reactivity of the sulfane sulfur atom(s) in polysulfides can vary with the pH, temperature, and the presence of any coexisting chemicals that accelerate or suppress hydrolysis (Hamid et al., 2019; Kasamatsu et al., 2021). These results suggest that it is quite difficult to obtain a representative molecule for all endogenous reactive polysulfides. Therefore, we emphasize that the importance of reactive polysulfide profiling using these two novel methods for quantitative determination of the TPsC and RPsC, in combination with untargeted polysulfide omics analysis to identify the contents and compositions of polysulfides.

### 3.4 Reactive Polysulfide Profiling in Vegetables

Using the above-mentioned methods, we analyzed the reactive pol-ysulfide profiles of 22 types of vegetables, including the Allium and Brassica vegetables, namely onions, garlic, and broccoli, and their cor-responding TSC, TPsC, and RPsC are summarized in Table 1. The ac-curacy and reproducibility of the results was confirmed by repeating at least independent three experiments. As indicated, the highest TSC was observed for Brassica rapa var. perviridis (Komatsuna), followed by Raphanus sativus var. longipinnatus (Daikon), broccoli, and Allium tuberosum (Chinese chive). Importantly, the TSC values obtained for the four vegetables and the garlic sample were significantly higher than those reported in previous studies (Aghajanzadeh, Hawkesford, & De Kok, 2014; Albrecht, Schafer, & Zottola, 1991; Doleman et al., 2017; Perner, Schwarz, Krumbein, & George, 2011), thereby suggesting that our acid-circulating decomposition system is a useful method for the precise detection of the TSC in vegetables. However, it is known that the soil sulfur fertility can alter the concentration and composition of the organosulfur compounds found in various vegetables, such as onions, garlic, komatsuna (Aghajanzadeh et al., 2014; Randle et al., 1995; Ueda, Kawajiri, Miyamura, & Miyajima, 1991). In fact, the obtained TSC values for our onion and garlic specimens showed quantitative simi-larities and differences with previous reports on these vegetables (Randle et al., 1995; Ueda et al., 1991), and so further studies are necessary to investigate the impacts of the sulfur fertility and seasonal variations on the TSC of vegetables using our acid-circulating decom-position system.

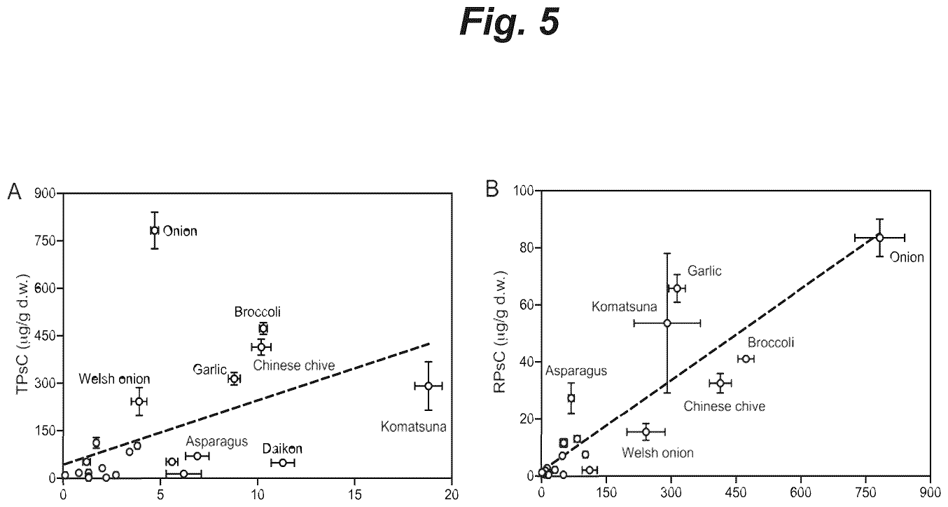

Subsequent quantification of the obtained TPsC revealed that the onion specimen contained the largest amount of polysulfides among the vegetables analyzed herein, accounting for 16.7% of the TSC, and this was followed by broccoli, Chinese chive, and garlic. Similarly, the highest RPsC was also detected in the onion sample, followed by garlic, komatsuna, and broccoli. To the best of our knowledge, this is the first report of quantitative reactive polysulfide profiles in fresh vegetables such as Allium and Brassica. Interestingly, FIG. 5A shows a moderate positive correlation between the TSC and TPsC (R²=0.2142), whereas FIG. 5B shows a strong positive correlation between the TPsC and RPsC (R²=0.7993). This difference can be explained, at least partially, by considering the large proportion of inorganic and/or organic sulfur-containing molecules that do not contain sulfane sulfur atom(s) (e.g., sulfate). Indeed, it has been reported that Brassica vegetables, such as cabbage, radish, and komatsuna, contain considerable amounts of sul-fates, accounting for 94, 74.7, and ˜50% of the total sulfur content, respectively (Aghajanzadeh et al., 2014; Castro et al., 2004; Doleman et al., 2017), whereas the sulfate levels in garlic and brown onions ac-count for 1.0 and 8.6% of the total sulfur, respectively (Doleman et al., 2017).

Kim et al. reported that an aqueous extract of garlic exhibited a higher antioxidant activity than that of onion, which correlates with the greater content of organosulfur compounds in the former, wherein example compounds include the γ-glutamyl peptides, S-alk(en)yl-cys-teines, and S-alk(en)yl-L-cysteine sulfoxides (S. Kim et al., 2018). Miller et al. reported similar results using garlic and onion extracts prepared using 50% methanol/water solvent mixtures (Miller, Rigelhof, Mar-quart, Prakash, & Kanter, 2000). However, in contrast, Nuutila et al. reported that a methanol extract of onion clearly exhibited a superior radical scavenging activity than that of garlic (Nuutila, Puupponen-Pimia\ddot{ }, Aarni, & Oksman-Caldentey, 2003). These discrepancies can be explained at least partially by considering the compositional differences in the organosulfur compounds of the onion and garlic specimens following extraction with different solvents. The biotransformation of organosulfur compounds has been demonstrated to be facilitated by alliinase to produce sulfenic acids in Alliums using S-alk(en)yl-L-cysteine-sulfoxides as substrates. Subsequently, the formed sulfenic acids condense and rearrange to produce the corresponding lipophilic polysulfides (Corzo-Martínez et al., 2007). Thus, extraction using high concentrations of organic solvents, such as methanol, ethanol, and acetone, can effectively deactivate alliinase and limit the transformation of S-alk(en)yl-L-cysteine-sulfoxides. This restricts the compositional and structural variations of organosulfur compounds, particularly, the lipo-philic polysulfides (i.e., DAS3). It has been reported that the major lipophilic polysulfide present in garlic oil (i.e., DAS3) can effectively scavenge the superoxide anions generated by a nonenzymatic system (i. e., phenazine methosulfate/NADH), wherein the scavenging activity increases with the number of sulfur atom, i.e., DAS3>DAS2>DAS (Kim, Chang, Kim, Chang, & Chun, 2006). Furthermore, various studies based on cell and animal experiments have shown that DAS3 exhibits a more potent anticancer effect than DAS2 (Puccinelli & Stan, 2017), further suggesting that the number of tethered sulfur atom(s) in a polysulfide may affect its biological function. Furthermore, several studies have demonstrated that the nucleophilic substituents present on poly-sulfides and their resulting lipophilicities may play important roles in determining their bioactivities (Casella, Leonardi, Melai, Fratini, & Pistelli, 2013; Iitsuka et al., 2010). In the present study, quantitative analyses revealed that the TPsC and RPsC values of freshly crushed onions were larger than the corresponding values for garlic. Although further careful studies are required to assess the correlation between the reactive polysulfide profiles and biological activities of these vegetables, it is expected that a dietary intake of vegetables containing abundant polysulfides, especially highly reactive ones, may promote health benefits. Hence, reactive polysulfide profiling could lead to the optimization of processing conditions for evaluating the beneficial health effects of vegetables and their derivatives, ultimately lead to the development of more effective preventive and therapeutic approaches for various diseases.

TABLE 1
The [symbol] value determined for the analyzed vegetables.

| | [symbol] | [symbol] | [symbol] |
|---|---|---|---|
| Species (Name) | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] var. [symbol] | [symbol] ± [symbol] | [symbol] ± [symbol] | [symbol] ± [symbol] |
| ( [symbol] ) | ( [symbol] ) | ( [symbol] ) | ( [symbol] ) |
| [symbol] var. | [symbol] ± [symbol] | [symbol] ± [symbol] | [symbol] ± [symbol] |
| [symbol] ( [symbol] ) | ( [symbol] ) | ( [symbol] ) | ( [symbol] ) |
| [symbol] var. | [symbol] ± [symbol] | [symbol] ± [symbol] | [symbol] ± [symbol] |
| [symbol] (Cabbage) | ( [symbol] ) | ( [symbol] ) | ( [symbol] ) |
| [symbol] var. | [symbol] ± [symbol] | [symbol] ± [symbol] | [symbol] ± [symbol] |
| [symbol] ( [symbol] ) | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] (Cucumber) | [symbol] ± [symbol] | [symbol] ± [symbol] | [symbol] ± [symbol] |
| | ( [symbol] ) | ( [symbol] ) | ( [symbol] ) |
| [symbol] (Lettuce) | [symbol] ± [symbol] | [symbol] ± [symbol] | [symbol] ± [symbol] |
| | ( [symbol] ) | ( [symbol] ) | ( [symbol] ) |
| [symbol] var. [symbol] | [symbol] ± [symbol] | [symbol] ± [symbol] | [symbol] ± [symbol] |
| [symbol] ( [symbol] ) | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |
| [symbol] (Carrot) | [symbol] ± [symbol] | [symbol] ± [symbol] | [symbol] ± [symbol] |
| | ( [symbol] ) | ( [symbol] ) | ( [symbol] ) |
| [symbol] var. | [symbol] ± [symbol] | [symbol] ± [symbol] | [symbol] ± [symbol] |
| [symbol] ( [symbol] pepper) | ( [symbol] ) | ( [symbol] ) | ( [symbol] ) |
| [symbol] var. | [symbol] ± [symbol] | [symbol] ± [symbol] | [symbol] ± [symbol] |
| [symbol] (Bell pepper) | ( [symbol] ) | ( [symbol] ) | ( [symbol] ) |
| [symbol] (Eggplant) | [symbol] ± [symbol] | [symbol] ± [symbol] | [symbol] ± [symbol] |
| | ( [symbol] ) | ( [symbol] ) | ( [symbol] ) |
| [symbol] (Tomato) | [symbol] ± [symbol] | [symbol] ± [symbol] | [symbol] ± [symbol] |
| | ( [symbol] ) | ( [symbol] ) | ( [symbol] ) |
| [symbol] (Spinach) | [symbol] ± [symbol] | [symbol] ± [symbol] | [symbol] ± [symbol] |
| | ( [symbol] ) | ( [symbol] ) | ( [symbol] ) |
| [symbol] ( [symbol] ) | [symbol] ± [symbol] | [symbol] ± [symbol] | [symbol] ± [symbol] |
| | ( [symbol] ) | ( [symbol] ) | ( [symbol] ) |

Data are presented as the mean ± SE (n − [symbol] ) d.w. = dry weight. [symbol]
indicates data missing or illegible when filed

The Brassica vegetables are known for their abundance of bioactive substances, and so the dietary intake of such substances and their breakdown products is desirable in the context of their potential anticancer, anti-inflammatory, and cardioprotective effects (Fuentes, Paredes-Gonzalez, & Kong, 2015; Miękus et al., 2020). Several previous studies have reported that the contents and compositions of the bioactive substances present in Brassica vegetables, such as broccoli and cabbage, are altered during germination and growth (Bellostas, Kachlicki, Sørensen, & Sørensen, 2007; Gu et al., 2012). However, little attention has been paid to the beneficial health effects of the Brassica polysulfides. In this study, quantitative analyses demonstrated that broccoli contained the highest amount of polysulfides among the Brassica vegetables examined herein; cabbage contained the second highest amount. Further studies are therefore required to investigate the relationship between the reactive polysulfide profiles and the germination and growth processes in these vegetables.

Studies focusing on the dietary intake of Alliums and Brassica vegetables have gained particular attention in recent years because of the cardioprotective functions of these species, which in turn have been attributed to the anti-inflammatory and antioxidant activities of their organosulfur compounds. Thus, to investigate the roles of such organosulfur compounds in the human body, various studies have examined their bioaccessibility and bioavailability (Putnik et al., 2019). However, it has been reported that organosulfur compounds can be metabolized after absorption (Liang, Wu, Wong, & Huang, 2015), and the sulfane sulfur atom(s) in polysulfides are known to be easily transferred to endogenous sulfur compounds, such as GSH, cysteine, and protein thiols. In addition, a previous study showed that the organosulfur compounds in garlic (e.g., allicin, DAS, DAS2, and DAS3) remain 66-100% intact under in vitro gastrointestinal digestion, although they also reported that only allicin and DAS are absorbed in the intestine (<2% each) (Torres-Palazzolo et al., 2018). Owing to these chemical properties of the polysulfides, it remains challenging to trace the overall sulfur transfer pathway from exogenous polysulfides to endogenous molecules. We therefore expect that our methods for quantitative determination of the TPsC and RPsC, in combination with quantitative metabolomics for target organosulfur compounds including polysulfides, will render it possible to gain further insight into the sulfur transfer pathway. Moreover, identification of the products formed by chemical or sulfur transfer reactions is also necessary. In this context, we recently developed a novel alkylating agent, namely N-iodoacetyl L-tyrosine methyl ester, by which unstable polysulfides, such as glutathione hydrotrisulfide and cysteine hydrotrisulfide, can be converted into stable derivatives without any considerable artificial decomposition (Kasamatsu et al., 2021). In preliminary experiments carried out using this novel detection probe, some unknown polysulfide candidates were detected in various vegetables, including garlic, onions, and broccoli. Future studies will investigate the use of this agent in greater detail. It is also necessary to carefully investigate that the chemical properties of sulfane sulfur atom(s) in the novel polysulfides using the alkaline/reductive sulfur elimination protocol and the NEM sulfur abstraction method developed herein, because the reactivity of sulfane sulfur atom(s) may differ by the molecular structures.

### 4. CONCLUSIONS

In the current study, we report that alkaline/reductive sulfur elimi-nation protocol, NEM sulfur abstraction method, and acid-circulating decomposition system are easy, convenient, and reliable methods to quantify of the total polysulfide content (TPsC), reactive polysulfide content (RPsC), and total sulfur content (TSC), respectively. Quantita-tive analyses revealed that onions contained the largest amount of pol-ysulfides among the 22 types of fresh vegetables analyzed, followed by broccoli, Chinese chive, and garlic. A strong positive correlation was observed between the TPsC and RPsC, whereas the TSC was only found to have a moderate correlation with the TPsC. Although further studies are required to investigate the relationship between the reactive poly-sulfide profiles and their biological functions, our results suggest that reactive polysulfide profiling can be considered a novel criterion for developing optimal processing conditions and evaluating the beneficial health benefits of vegetables and their derivatives. Ultimately, this may lead to the development of more effective preventive and therapeutic approaches for diseases based on vegetable-derived polysulfides.

### APPENDIX A. SUPPLEMENTARY DATA

Supplementary data to this article can be found online at https://doi.org/10.1016/j.foodchem.2023.135610.

### REFERENCES

All the articles listed below and their disclosures are incorporated in this application by reference.
Aghajanzadeh, T., Hawkesford, M. J., & De Kok, L. J. (2014). The significance of glucosinolates for sulfur storage in Brassicaceae seedlings. Frontiers in Plant Science, 5, 704. https://doi.org/10.3389/fpls.2014.00704
Akaike, T., Ida, T., Wei, F. Y., Nishida, M., Kumagai, Y., Alam, M. M., . . . . Motohashi, H. (2017). Cysteinyl-tRNA synthetase governs cysteine polysulfidation and mitochondrial bioenergetics. Nature Communications, 8(1), 1177. https://doi.org/10.1038/s41467-017-01311-y
Albrecht, J. A., Schafer, H. W., & Zottola, E. A. (1991). Sulfhydryl and ascorbic acid relationships in selected vegetables and fruits. Journal of Food Science, 56(2), 427-430. https://doi.org/10.1111/j.1365-2621.1991.tb05296.x
Ali, M. W., Zoltai, S. C., & Radford, F. G. (1988). A comparison of dry and wet ashing methods for the elemental analysis of peat. Canadian Journal of Soil Science, 68(2), 443-447. https://doi.org/10.4141/cjss88-041
Anderson, L.-O., & Berg, G. (1969). Hydrolysis of disulfide bonds in weakly alkaline media. I. Oxidized glutathione. Biochimica et Biophysica Acta (BBA)—General Subjects, 192(3), 534-536. https://doi.org/10.1016/0304-4165(69)90406-1.
Barayeu, U., Schilling, D., Eid, M., Xavier Da Silva, T. N., Schlicker, L., Mitreska, N., . . . . Dick, T. P. (2022). Hydropersulfides inhibit lipid peroxidation and ferroptosis by scavenging radicals. Nature Chemical Biology. https://doi.org/10.1038/s41589-022-01145-w
Bellostas, N., Kachlicki, P., Sørensen, J. C., & Sørensen, H. (2007). Glucosinolate profiling of seeds and sprouts of B. oleracea varieties used for food. Scientia Horticulturae, 114(4), 234-242. https://doi.org/10.1016/j.scienta.2007.06.015.
Bogd\acute{ }andi, V., Ida, T., Sutton, T. R., Bianco, C., Ditroi, T., Koster, G., . . . . Nagy, P. (2019). Speciation of reactive sulfur species and their reactions with alkylating agents: Do we have any clue about what is present inside the cell? British Journal of Pharmacology, 176(4), 646-670. https://doi.org/10.1111/bph.14394
Casella, S., Leonardi, M., Melai, B., Fratini, F., & Pistelli, L. (2013). The role of diallyl sulfides and dipropyl sulfides in the in vitro antimicrobial activity of the essential oil of garlic, Allium sativum L., and Leek, Allium porrum L. Phytotherapy Research, 27(3), 380-383. https://doi.org/10.1002/ptr.4725
Castro, A., Aires, A., Rosa, E., Bloem, E., Stulen, I., & De Kok, L. J. (2004). Distribution of glucosinolates in Brassica oleracea cultivars. Phyton; annales rei botanicae, 44, 133-143.
Corzo-Martínez, M., Corzo, N., & Villamiel, M. (2007). Biological properties of onions and garlic. Trends in Food Science & Technology, 18(12), 609-625. https://doi.org/10.1016/j.tifs.2007.07.011.
Doleman, J. F., Grisar, K., Van Liedekerke, L., Saha, S., Roe, M., Tapp, H. S., & Mithen, R. F. (2017). The contribution of alliaceous and cruciferous vegetables to dietary sulphur intake. Food Chemistry, 234, 38-45. https://doi.org/10.1016/j.foodchem.2017.04.098.
Fuentes, F., Paredes-Gonzalez, X., & Kong, A.-N.-T. (2015). Dietary glucosinolates sulforaphane, phenethyl isothiocyanate, indole-3-carbinol/3,3′-diindolylmethane: Antioxidative stress/inflammation, nrf2, epigenetics/epigenomics and in vivo cancer chemopreventive efficacy. Current Pharmacology Reports, 1(3), 179-196. https://doi. org/10.1007/s40495-015-0017-y
Gu, Y., Guo, Q., Zhang, L., Chen, Z., Han, Y., & Gu, Z. (2012). Physiological and biochemical metabolism of germinating broccoli seeds and sprouts. Journal of Agricultural and Food Chemistry, 60(1), 209-213. https://doi.org/10.1021/jf203599v
Hamid, H. A., Tanaka, A., Ida, T., Nishimura, A., Matsunaga, T., Fujii, S., . . . . Akaike, T. (2019). Polysulfide stabilization by tyrosine and hydroxyphenyl-containing derivatives that is important for a reactive sulfur metabolomics analysis. Redox Biology, 21, Article 101096. https://doi.org/10.1016/j.redox.2019.101096
Hill, C. R., Shafaci, A., Balmer, L., Lewis, J. R., Hodgson, J. M., Millar, A. H., & Blekkenhorst, L. C. (2022). Sulfur compounds: From plants to humans and their role in chronic disease prevention. Critical Reviews in Food Science and Nutrition, April 5, 1-23. https://doi.org/10.1080/10408398.2022.2057915.
Høj, P. B., & Møller, B. L. (1987). Acid-labile sulfide and zero-valence sulfur in plant extracts containing chlorophyll and ionic detergents. Analytical Biochemistry, 164(2), 307-314. https://doi.org/10.1016/0003-2697(87) 90498-2.
Ida, T., Sawa, T., Ihara, H., Tsuchiya, Y., Watanabe, Y., Kumagai, Y., . . . . Akaike, T. (2014). Reactive cysteine persulfides and S-polythiolation regulate oxidative stress and redox signaling. Proceedings of the National Academy of Sciences of the United States of America, 111(21), 7606-7611. https://doi.org/10.1073/pnas.1321232111.
Iitsuka, Y., Tanaka, Y., Hosono-Fukao, T., Hosono, T., Seki, T., & Ariga, T. (2010). Relationship between lipophilicity and inhibitory activity against cancer cell growth of nine kinds of alk(en)yl trisulfides with different side chains. Oncology research, 18(11-12), 575-582. https://doi.org/10.3727/096504010x12767359113965
Ishigami, M., Hiraki, K., Umemura, K., Ogasawara, Y., Ishii, K., & Kimura, H. (2009). A source of hydrogen sulfide and a mechanism of its release in the brain. Antioxidants & Redox Signaling, 11(2), 205-214. https://doi.org/10.1089/ars.2008.2132
Kasamatsu, S., Ida, T., Koga, T., Asada, K., Motohashi, H., Ihara, H., & Akaike, T. (2021). High-precision sulfur metabolomics innovated by a new specific probe for trapping reactive sulfur species. Antioxidants & redox signaling, 34(18), 1407-1419. https://doi.org/10.1089/ars.2020.8073
Kasamatsu, S., Kakihana, Y., Koga, T., Yoshioka, H., & Ihara, H. (2020). Generation of rat monoclonal antibody to detect hydrogen sulfide and polysulfides in biological samples. Antioxidants, 9(11), 1160. https://doi.org/10.3390/antiox9111160
Kim, J.-M., Chang, H. J., Kim, W.-K., Chang, N., & Chun, H. S. (2006). Structure-activity relationship of neuroprotective and reactive oxygen species scavenging activities for allium organosulfur compounds. Journal of Agricultural and Food Chemistry, 54(18), 6547-6553. https://doi.org/10.1021/jf060412c
Kim, S., Kim, D.-B., Jin, W., Park, J., Yoon, W., Lee, Y., . . . . Yoo, M. (2018). Comparative studies of bioactive organosulphur compounds and antioxidant activities in garlic (Allium sativum L.), elephant garlic (Allium ampeloprasum L.) and onion (Allium cepa L.). Natural Product Research, 32(10), 1193-1197. https://doi.org/10.1080/14786419.2017.1323211
Liang, D., Wu, H., Wong, M. W., & Huang, D. (2015). Diallyl trisulfide is a fast H₂S donor, but diallyl disulfide is a slow one: the reaction pathways and intermediates of glutathione with polysulfides. Organic Letters, 17(17), 4196-4199. https://doi.org/10.1021/acs.orglett.5b01962
Locatelli, D. A., Nazareno, M. A., Fusari, C. M., & Camargo, A. B. (2017). Cooked garlic and antioxidant activity: Correlation with organosulfur compound composition.
Food Chemistry, 220, 219-224. https://doi.org/10.1016/j.foodchem.2016.10.001. Marutani, E., Morita, M., Hirai, S., Kai, S., Grange, R. M. H., Miyazaki, Y., . . . . Ichinose, F. (2021). Sulfide catabolism ameliorates hypoxic brain injury. Nature Communications, 12(1), 3108. https://doi.org/10.1038/s41467-021-23363-x
Miękus, N., Marszałek, K., Podlacha, M., Iqbal, A., Puchalski, C., & S\acute{ }wiergiel, A. H. (2020). Health benefits of plant-derived sulfur compounds, glucosinolates, and organosulfur compounds. Molecules, 25(17), 3804. https://doi.org/10.3390/molecules25173804
Miller, H. E., Rigelhof, F., Marquart, L., Prakash, A., & Kanter, M. (2000). Antioxidant content of whole grain breakfast cereals, fruits and vegetables. Journal of the American College of Nutrition, 19 (sup3), 312S-319S. https://doi.org/10.1080/07315724.2000.10718966
Nuutila, A. M., Puupponen-Pimia\ddot{ }, R., Aarni, M., & Oksman-Caldentey, K.-M. (2003). Comparison of antioxidant activities of onion and garlic extracts by inhibition of lipid peroxidation and radical scavenging activity. Food Chemistry, 81(4), 485-493. https://doi.org/10.1016/S0308-8146(02)00476-4.
Olson, K. R., DeLeon, E. R., & Liu, F. (2014). Controversies and conundrums in hydrogen sulfide biology. Nitric Oxide, 41, 11-26. https://doi.org/10.1016/j.niox.2014.05.0 12.
Perner, H., Schwarz, D., Krumbein, A., & George, E. (2011). Influence of sulfur supply, ammonium nitrate ratio, and arbuscular mycorrhizal colonization on growth and composition of Chinese chive. Scientia Horticulturae, 130(3), 485-490. https://doi.org/10.1016/j.scienta.2011.07.028.
Puccinelli, M. T., & Stan, S. D. (2017). Dietary bioactive diallyl trisulfide in cancer prevention and treatment. International Journal of Molecular Sciences, 18(8), 1645. https://doi.org/10.3390/ijms18081645
Putnik, P., Gabri\acute{ }c, D., Roohinejad, S., Barba, F. J., Granato, D., Mallikarjunan, K., . . . . Bursa\acute{ }c Kova ̌cevi\acute{ }c, D. (2019). An overview of organosulfur compounds from Allium spp.: From processing and preservation to evaluation of their bioavailability, antimicrobial, and anti-inflammatory properties. Food Chemistry, 276, 680-691. https://doi.org/10.1016/j.foodchem.2018.10.068.
Randle, W. M., Lancaster, J. E., Shaw, M. L., Sutton, K. H., Hay, R. L., & Bussard, M. L. (1995). Quantifying onion flavor compounds responding to sulfur fertility-sulfur increases levels of alk(en)yl cysteine sulfoxides and biosynthetic intermediates. Journal of the American Society for Horticultural Science, 120(6), 1075-1081. https://doi.org/10.21273/jashs. 120.6.1075.
Sawa, T., Takata, T., Matsunaga, T., Ihara, H., Motohashi, H., & Akaike, T. (2022). Chemical biology of reactive sulfur species: Hydrolysis-driven equilibrium of polysulfides as a determinant of physiological functions. Antioxidants & Redox Signaling, 36(4-6), 327-336. https://doi.org/10.1089/ars.2021.0170
Shinohara, K., & Padis, K. E. (1936). The determination of thiol and disulfide compounds, with special reference to cysteine and cystine: Vii. Application of the modified phospho-18-tungstic acid method for the determination of cysteine, cystine, and ascorbic acid in urine. Journal of Biological Chemistry, 112(2), 709-721. https://doi. org/10.1016/S0021-9258(18)74953-3.
Tocmo, R., Liang, D., Lin, Y., & Huang, D. (2015). Chemical and biochemical mechanisms underlying the cardioprotective roles of dietary organopolysulfides. Frontiers in Nutrition, 2, 1. https://doi.org/10.3389/fnut.2015.00001
Torres-Palazzolo, C., Ramirez, D., Locatelli, D., Manucha, W., Castro, C., & Camargo, A. (2018). Bioaccessibility and permeability of bioactive compounds in raw and cooked garlic. Journal of Food Composition and Analysis, 70, 49-53. https://doi.org/10.1016/j.jfca.2018.03.008
Ueda, Y., Kawajiri, H., Miyamura, N., & Miyajima, R. (1991). Content of some sulfur-containing components and free amino acids in various strains of garlic. Nippon Shokuhin Kogyo Gakkaishi, 38(5), 429-434. https://doi.org/10.3136/nskkk1962.38.429
Zhou, W., Yang, S., & Wang, P. G. (2017). Matrix effects and application of matrix effect factor. Bioanalysis, 9(23), 1839-1844. https://doi.org/10.4155/bio-2017-0214

What is claimed is:

1. Method for quantitative determination of total polysulfide content (TPsC) and reactive polysulfide content (RPsC), comprising
a step of using liquid chromatography-electrospray ionization-tandem mass spectrometry.
